(19) United States
(12) Patent Application Publication
(10) Pub. No.: US 2022/0071941 A1
(43) Pub. Date: Mar. 10, 2022

(54) THERAPIES FOR SQUAMOUS CELL CARCINOMAS

(71) Applicant: Kura Oncology, Inc., San Diego, CA (US)

(72) Inventors: Antonio Gualberto, Boston, MA (US); Catherine Rose Scholz, North Reading, MA (US); Vishnu S. Mishra, San Diego, CA (US)

(21) Appl. No.: 17/416,086

(22) PCT Filed: Dec. 20, 2019
(86) PCT No.: PCT/US2019/067843

§ 371 Date: Jun. 18, 2021

Related U.S. Application Data
(60) Provisional application No. 62/844,987, filed on May 8, 2019.
(60) Provisional application No. 62/784,092, filed on Dec. 21, 2018.

(51) Int. Cl. A61K 31/223; A61K 45/06; A61K 31/4709; A61K 31/4545; A61K 31/365; A61K 31/045; A61K 31/496; A61K 31/55; A61K 31/5513; A61P 35/00

(52) U.S. Cl. CPC A61K 31/223; A61K 45/06; A61K 31/4709; A61K 31/4545; A61P 35/00; A61K 31/045; A61K 31/496; A61K 31/55; A61K 31/5513; A61K 31/365

(57) ABSTRACT

The present invention relates to the field of cancer therapy. Specifically, provided are methods of neoadjuvant therapies or adjuvant therapies to treat squamous cell carcinoma in a subject with a farnesyltransferase inhibitor (FTI) that include determining whether the subject is likely to be responsive to the FTI treatment based on the H-Ras mutant allele frequency.

### FIELD

The present invention relates to the field of cancer therapy. In particular, provided herein are methods of uses of farnesyltransferase inhibitors (“FTI”) in neoadjuvant therapies and adjuvant therapies for squamous cell carcinomas (“SCC”).

### BACKGROUND

Various therapeutic options, including surgery, radiation and systemic therapies, are available for patients diagnosed with SCC. However, not all SCC patients are candidates for these therapies for a variety of reasons. For example, large tumor volume may prevent certain patients from receiving surgery or radiation treatment. In addition, it may be advisable to shrink the size of a tumor prior to surgical removal. Thus, neoadjuvant therapies and adjuvant therapies that convert untreatable SCC to treatable SCC or that may provide clinical benefit to SCC patients prior to or after surgery represent unmet needs. The methods and compositions provided herein meet these needs and provide other related advantages.

### SUMMARY

Provided herein are methods of neoadjuvant therapies and adjuvant therapies in a subject having an H-Ras mutant SCC. In some embodiments, provided herein are methods of treating a subject having an H-Ras mutant SCC in conjunction with surgery, comprising administering a therapeutically effective amount of an FTI to the subject, wherein the SCC has an H-Ras mutant allele frequency (AF) that is greater than 20%.

In some embodiments, the H-Ras mutant AF is greater than 25%, 30%, 35%, 40%, 45%, 50%, 55%, or 60%.

In some embodiments, the SCC has an amino acid substitution in H-Ras at a codon selected from a group consisting of G12, G13, Q61, Q22, K117, A146, and any combination thereof.

In some embodiments, the SCC does not have K-Ras mutation or N-Ras mutation.

In some embodiments, provided herein are neoadjuvant therapies comprising administering FTI prior to surgery, wherein the SCC has an H-Ras mutant allele frequency (AF) that is greater than 20%, 25%, 30%, 35%, 40%, 45%, 50%, 55%, or 60%.

In some embodiments, the neoadjuvant therapy provided herein reduces tumor burden in the subject by at least 20%, at least 30%, at least 40%, at least 50%, at least 60%, at least 70%, at least 80%, at least 90%, or at least 95%. In some embodiments, the neoadjuvant therapy improves the efficacy of the surgery. In some embodiments, the neoadjuvant therapy converts the SCC from inoperable to operable. In some embodiments, the neoadjuvant therapy reduces the risk of recurrence after the surgery.

In some embodiments, provided herein are adjuvant therapies comprising administering FTI after surgery, wherein the SCC has an H-Ras mutant allele frequency (AF) that is greater than 20%, 25%, 30%, 35%, 40%, 45%, 50%, 55%, or 60%. In some embodiments, the adjuvant therapy improves the efficacy of the surgery. In some embodiments, the adjuvant therapy reduces the risk of recurrence after the surgery.

In some embodiments, the SCC is head and neck SCC (HNSCC), lung SCC (LSCC), thyroid SCC (TSCC), esophageal SCC (ESCC), bladder SCC (BSCC), urothelial carcinoma (UC), cutaneous SCC (CuSCC), cervical SCC (CSCC), vulvar SCC (VSCC) or penile SCC (PSCC). In some embodiments, the SCC is HNSCC. In some embodiments, the HNSCC is HNSCC of the trachea. In some embodiments, the HNSCC is HNSCC of the maxilla. In some embodiments, the HNSCC is HNSCC of the oral cavity.

In some embodiments, the SCC is human papillomavirus (HPV)-negative. In some embodiments, the SCC is at an advanced stage or metastatic. In some embodiments, the SCC is relapsed. In some embodiments, the SCC is refractory. In some embodiments, the subject having SCC is newly diagnosed. In some embodiments, the subject having SCC has not received any prior treatment for SCC.

In some embodiments, methods provided herein further comprise determining the H-Ras mutant AF in a sample from the subject. In some embodiments, the H-Ras mutant AF is determined by sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay.

In some embodiments, the sample is a tissue biopsy. In some embodiments, the sample is a tumor biopsy. In some embodiments, the sample is a blood sample. The blood sample can contain tumor DNA. In some embodiments, the sample is isolated cells.

In some embodiments, the FTI used in the methods provided herein is selected from the group consisting of tipifarnib, lonafarnib, arglabin, perrilyl alcohol, L778123, L739749, FTI-277, L744832, CP-609,754, 8208176, AZD3409, and BMS-214662. In some embodiments, the FTI is tipifarnib. In some embodiments, the FTI is lonafarnib. In some embodiments, the FTI is BMS-214662.

In some embodiments, the FTI is administered at a dose of 0.05-500 mg/kg body weight. In some embodiments, the FTI is administered at a daily dose of 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg.

In some embodiments, the FTI is administered twice a day. In some embodiments, the FTI is administered at a dose of 100-1000 mg twice a day. In some embodiments, the FTI is administered at a dose of 100 mg, 200 mg, 300 mg, 400 mg, 500 mg, 600 mg, 800 mg, 900 mg, or 1000 mg twice a day.

In some embodiments, methods provided herein comprises administering the FTI for a period of one to seven days. In some embodiments, the FTI is administered on days 1-7 of a 28-day treatment cycle. In some embodiments, the FTI is administered on days 1-7 and 15-21 of a 28-day treatment cycle. In some embodiments, the FTI is administered on days 1-21 of a 28-day treatment cycle. In some embodiments, the FTI is administered for at least 3 cycles, at least 6 cycles, at least 9 cycles, or at least 12 cycles.

### BRIEF DESCRIPTION OF THE FIGURE

FIG. 1: Waterfall plots based on analysis of subjects with HRAS VAF ≥20% and serum albumin ≥3.5 g/dL or HRAS VAF ≥35% enrolled in stages 1, 2 or in the HNSCC extension cohort. Subjects were treated either with tipifarnib or the last prior therapy received.

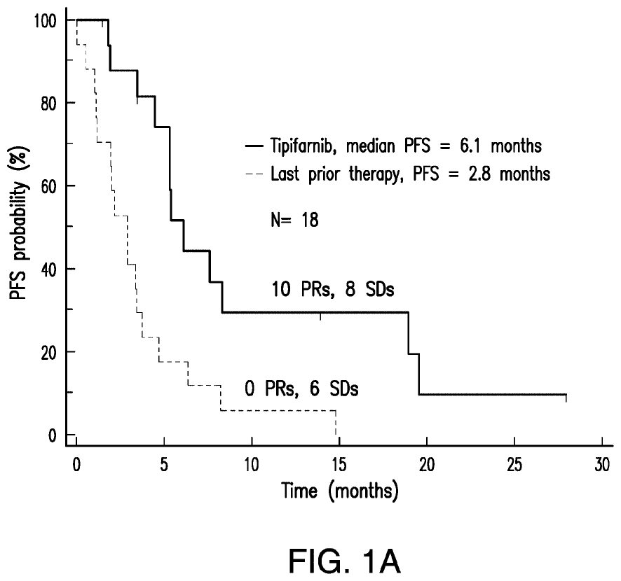

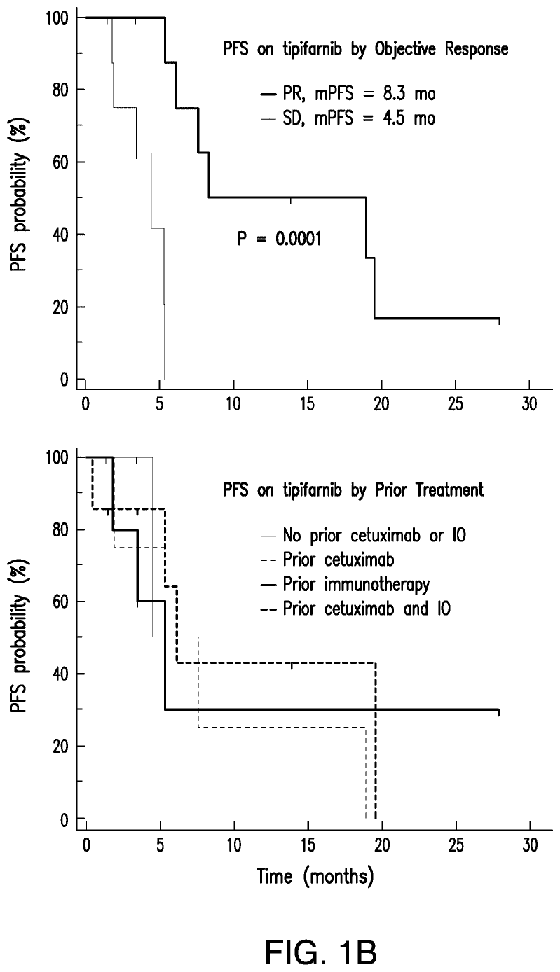

### DETAILED DESCRIPTION

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. A subject can be a human.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. Exemplary samples include lymph node, whole blood, partially purified blood, serum, plasma, bone marrow, and peripheral blood mononuclear cells (“PBMC”). A sample also can be a tissue biopsy. Exemplary samples also include cell lysate, a cell culture, a cell line, a tissue, oral tissue, gastrointestinal tissue, an organ, an organelle, a biological fluid, a blood sample, a urine sample, a skin sample, and the like.

As used herein, the terms “treat,” “treating,” and “treatment,” when used in reference to a cancer patient, can refer to an action that reduces the severity of the cancer, or retards or slows the progression of the cancer, including (a) inhibiting the cancer growth, or arresting development of the cancer, and (b) causing regression of the cancer, or delaying or minimizing one or more symptoms associated with the presence of the cancer. For example, “treating” a cancer, such as a HRas overexpressing SCC in a subject refers to an action inhibiting the cancer growth in the subject or causing an HRas overexpressing SCC in a subject to regress in size.

As used herein, the term “neoadjuvant therapy” refers to a treatment that is administered before the surgery, and the term “adjuvant therapy” refers to a treatment administered during or after the surgery. These multimodal treatment approaches can increase the effectiveness of the main therapy and/or minimize the adverse effects patients experience during main therapy.

As used herein, the term “HRas mutation” refers to an activating mutation in an HRAS gene or HRas protein. An HRas mutation can refer to either a genetic alternation in the DNA sequence of the HRAS gene that results in constitutive activation of the corresponding HRas protein, or the alteration in the amino acid sequence of an HRas protein that results in its activation. Thus, the term “HRas mutation” as used herein does not include an alteration in a HRAS gene that does not result in the activation of the HRAS protein, or an alternation of a HRas protein sequence that does not lead to its activation. Accordingly, a sample or a subject that does not have any “HRas mutation” as used herein can still have a mutation in the HRAS gene that does not affect the activity of the HRas protein or a mutation that impairs the activity of the HRas protein, or have a mutation in an HRAS protein that does not affect its activity or a mutation that impairs its activity. A sample or a subject can have multiple copies of the HRAS gene. A sample or a subject can also have both wild type and mutant HRas proteins. As used herein, a sample or a subject having an HRas mutation can also have a copy of wild type HRAS gene and/or the wild type HRas protein. A sample or a subject that is determined to “have wild type HRAS,” as used herein, refers to the sample or subject that only has the wild type HRAS gene and the wild type HRas protein, and no HRas mutation.

As used herein, the term “allele frequency” or “AF” refers to the incidence of a gene variant in a population of cells. Alleles are variant forms of a gene that are located at the same position, or genetic locus, on a chromosome. An allele frequency is calculated by dividing the number of times the allele of interest is observed in a population of cells by the total number of copies of all the alleles at that particular genetic locus in the population. An allele frequency of a particular gene mutation can refer to the amount of DNA present in a sample that contains the mutant allele over the total amount of DNA present in a sample, expressed as a percentage.

As used herein, the term “administer,” “administering,” or “administration” refers to the act of delivering, or causing to be delivered, a compound or a pharmaceutical composition to the body of a subject by a method described herein or otherwise known in the art. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

As used herein, the term “selecting” and “selected” in reference to a subject is used to mean that a particular subject is specifically chosen from a larger group of subjects on the basis of (due to) the particular subject meeting a predetermined criterion or a set of predetermined criteria, e.g., having certain HRas expression than a reference level. Similarly, “selectively treating” a subject refers to providing treatment to a subject meeting a predetermined criterion or a set of predetermined criteria. Similarly, “selectively administering” refers to administering a drug to a subject meeting a predetermined criterion or a set of predetermined criteria. By selecting, selectively treating and selectively administering, it is meant that a subject having SCC is delivered a personalized therapy based on the subject's biology, rather than being delivered a standard treatment regimen based solely on having SCC.

As used herein, the term “therapeutically effective amount” of a compound when used in connection with a disease or disorder refers to an amount sufficient to provide a therapeutic benefit in the treatment of the disease or disorder or to delay or minimize one or more symptoms associated with the disease or disorder. The disease or disorder refers can be SCC. A therapeutically effective amount of a compound means an amount of the compound that when used alone or in combination with other therapies, would provide a therapeutic benefit in the treatment or management of the disease or disorder. The term encompasses an amount that improves overall therapy, reduces or avoids symptoms, or enhances the therapeutic efficacy of another therapeutic agent. The term also refers to the amount of a compound that sufficiently elicits the biological or medical response of a biological molecule (e.g., a protein, enzyme, RNA, or DNA), cell, tissue, system, animal, or human, which is being sought by a researcher, veterinarian, medical doctor, or clinician.

### A. Methods

Provided herein are adjuvant therapies or neoadjuvant therapies for squamous cell carcinoma (SCC). In some embodiments, methods provided herein include treating a subject having an HRas mutant SCC in conjugation with surgery using a farnesyltransferase inhibitor (FTI). The methods provided herein are based, in part, on the discovery that SCC patients with various levels of HRas mutant AF respond differently to an FTI neoadjuvant therapy or adjuvant therapy, and that the clinical benefits of an FTI neoadjuvant therapy or adjuvant therapy are associated with the HRas mutant AF. For example, the methods provided herein are based on the discovery that SCC patients having HRas mutant AF that is greater than a reference level are likely responsive to an FTI neoadjuvant therapy or adjuvant therapy, and selection of a SCC patient population having HRas mutant AF that is greater than a reference level can increase the overall response rate of an FTI neoadjuvant therapy or adjuvant therapy.

The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib.

Provided herein are methods of treating a subject having an HRas mutant SCC in conjunction with surgery, comprising administering a therapeutically effective amount of an FTI to the subject, wherein the SCC has an HRas mutant AF that is greater than a reference level. Provided herein are also methods for increasing the responsiveness of an FTI neoadjuvant therapy or adjuvant therapy for SCC by selectively treating SCC patients having an HRas mutant AF that is greater than a reference level. Provided herein are also methods of predicting responsiveness of a subject having SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having SCC and administering an FTI neoadjuvant therapy or adjuvant therapy to the subject if the HRas mutant AF is greater than a reference level.

In some embodiments, the reference level is 35%. Accordingly, provided herein are methods of neoadjuvant therapy or adjuvant therapy for a subject having an HRas mutant SCC, comprising administering a therapeutically effective amount of an FTI to the subject, wherein the SCC has an HRas mutant AF that is greater 35%. Provided herein are also methods for increasing the responsiveness of an FTI neoadjuvant therapy or adjuvant therapy for SCC by selectively treating SCC patients having an HRas mutant AF that is greater than 35%. Provided herein are also methods of predicting responsiveness of a subject having SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having SCC and administering an FTI neoadjuvant therapy or adjuvant therapy to the subject if the HRas mutant AF is greater than 35%.

In some embodiments, the FTI neoadjuvant therapy or adjuvant therapy is selectively administered to a subject having SCC that has an HRas mutant AF greater than 20%, 25%, 30%, 40%, 45%, 50%, 55%, or 60%. In some embodiments, the FTI neoadjuvant therapy or adjuvant therapy is administered to a subject having SCC that has an HRas mutant AF greater than 20%. In some embodiments, the FTI neoadjuvant therapy or adjuvant therapy is administered to a subject having SCC that has an HRas mutant AF greater than 25%. In some embodiments, the FTI neoadjuvant therapy or adjuvant therapy is administered to a subject having SCC that has an HRas mutant AF greater than 30%. In some embodiments, the FTI neoadjuvant therapy or adjuvant therapy is administered to a subject having SCC that has an HRas mutant AF greater than 40%. In some embodiments, the FTI neoadjuvant therapy or adjuvant therapy is administered to a subject having SCC that has an HRas mutant AF greater than 45%. In some embodiments, the FTI neoadjuvant therapy or adjuvant therapy is administered to a subject having SCC that has an HRas mutant AF greater than 50%. In some embodiments, the FTI neoadjuvant therapy or adjuvant therapy is administered to a subject having SCC that has an HRas mutant AF greater than 55%. In some embodiments, the FTI neoadjuvant therapy or adjuvant therapy is administered to a subject having SCC that has an HRas mutant AF greater than 60%. In some embodiments, the FTI neoadjuvant therapy or adjuvant therapy is administered to a subject having SCC that has an HRas mutant AF greater than 65%. In some embodiments, the FTI neoadjuvant therapy or adjuvant therapy is administered to a subject having SCC that has an HRas mutant AF greater than 70%. In some embodiments, the FTI neoadjuvant therapy or adjuvant therapy is administered to a subject having SCC that has an HRas mutant AF greater than 75%. In some embodiments, the FTI neoadjuvant therapy or adjuvant therapy is administered to a subject having SCC that has an HRas mutant AF greater than 80%. In some embodiments, the FTI neoadjuvant therapy or adjuvant therapy is administered to a subject having SCC that has an HRas mutant AF greater than 85%. In some embodiments, the FTI neoadjuvant therapy or adjuvant therapy is administered to a subject having SCC that has an HRas mutant AF greater than 90%. In some embodiments, the FTI neoadjuvant therapy or adjuvant therapy is administered to a subject having SCC that has an HRas mutant AF greater than 90%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the HRas mutation comprises an amino acid substitution at a codon selected from a group consisting of G12, G13, Q61, Q22, K117, A146 and any combination thereof. In some embodiments, the HRas mutation is a mutation at a codon of G12. In some embodiments, the HRas mutation is a G12R substitution. The HRas mutation can be G12C, G12D, G12A, G12V, G12S, G12F, G12R, or G12N. In some embodiments, the HRas mutation can be, for example, a G12V substitution. In some embodiments, the HRas mutation is a mutation at a codon of G13. The HRas mutation can be G13A, G13C, G13V, G13D, G13R, G13S, or G13N. In some embodiments, the HRas mutation can be, for example, a G13C substitution, or a G13R substitution. In some embodiments, the HRas mutation is a mutation at a codon of Q61. The HRas mutation can be Q61E, Q61K, Q61H, Q61L, Q61P, or Q61R. In some embodiments, the HRas mutation can be, for example, a Q61L substitution, or a Q61R substitution. In some embodiments, the HRas mutation is a mutation at a codon of Q22. In some embodiments, the HRas mutation is a Q22K substitution. In some embodiments, the HRas mutation is a mutation at a codon of K117. In some embodiments, for example, the HRas mutation is a K117N or K117L substitution. In some embodiments, the HRas mutation is a mutation at a codon of A146. The HRas mutation can be A146V or A146P. In some embodiments, the HRas mutation is an A146P substitution. In some embodiments, the mutation can be a mutation at another codon that results in activation of HRas protein.

In some embodiments, the SCC does not have KRas mutation. In some embodiments, the SCC does not have NRas mutation. In some embodiments, the SCC does not have KRas mutation or NRas mutation.

FTI neoadjuvant therapies for SCC patients are provided herein. In some embodiments, methods provided herein include treating a subject having an H-Ras mutant SCC prior to surgery, comprising administering a therapeutically effective amount of an FTI to the subject, wherein the SCC has an H-Ras mutant AF that is greater than a reference level. In some embodiments, the subjects have untreatable SCC. In some embodiments, the subject has an inoperable SCC. In some embodiments, the neoadjuvant therapy provided herein converts an inoperable SCC to operable SCC in a subject.

In some embodiments, the neoadjuvant therapy downstages the SCC. In some embodiments, the neoadjuvant therapy provided herein reduces the tumor burden in the subject having SCC. In some embodiments, the neoadjuvant therapy provided herein reduces the tumor burden by at least 20%. In some embodiments, the neoadjuvant therapy provided herein reduces the tumor burden by at least 30%. In some embodiments, the neoadjuvant therapy provided herein reduces the tumor burden by at least 40%. In some embodiments, the neoadjuvant therapy provided herein reduces the tumor burden by at least 50%. In some embodiments, the neoadjuvant therapy provided herein reduces the tumor burden by at least 60%. In some embodiments, the neoadjuvant therapy provided herein reduces the tumor burden by at least 70%. In some embodiments, the neoadjuvant therapy provided herein reduces the tumor burden by at least 80%. In some embodiments, the neoadjuvant therapy provided herein reduces the tumor burden by at least 90%. In some embodiments, the neoadjuvant therapy provided herein reduces the tumor burden by at least 95%.

FTI adjuvant therapies for SCC patients are provided herein. In some embodiments, the FTI is administered after the main therapy. In some embodiments, methods provided herein include treating a subject having an H-Ras mutant SCC after the surgery, comprising administering a therapeutically effective amount of an FTI to the subject, wherein the SCC has an H-Ras mutant AF that is greater than a reference level.

In some embodiments, the FTI neoadjuvant therapy or adjuvant therapy provided herein optimizes surgical outcomes. In some embodiments, the FTI neoadjuvant therapy or adjuvant therapy provided herein improves the efficacy of the surgery. In some embodiments, the FTI neoadjuvant therapy or adjuvant therapy provided herein minimizes the adverse effects of the main therapy. In some embodiments, the FTI neoadjuvant therapy or adjuvant therapy provided herein reduces the risk of recurrence. In some embodiments, the FTI neoadjuvant therapy or adjuvant therapy provided herein improves the duration of clinical benefit of the main therapy.

In some embodiments, methods provided herein further include administering radiation therapy or chemotherapy to the subject. In some embodiments, methods provided herein further include administering radiation therapy to the subject. In some embodiments, methods provided herein further include administering chemotherapy to the subject.

SCC is an uncontrolled growth of abnormal cells arising from the squamous cells in the epidermis. Common types include head and neck SCC (HNSCC), lung SCC (LSCC), thyroid SCC, esophageal SCC, bladder SCC, urothelial carcinoma (UC), cervical SCC, penile SCC, vulvar SCC or cutaneous SCC. Human papillomavirus infection (HPV) has been associated with SCC development. In some embodiments, the methods provided herein are neoadjuvant therapies and/or adjuvant therapies for treating HNSCC. In some embodiments, the methods provided herein are neoadjuvant therapies and/or adjuvant therapies for treating LSCC. In some embodiments, the methods provided herein are neoadjuvant therapies and/or adjuvant therapies for treating thyroid SCC. In some embodiments, the methods provided herein are neoadjuvant therapies and/or adjuvant therapies for treating esophageal SCC. In some embodiments, the methods provided herein are neoadjuvant therapies and/or adjuvant therapies for treating bladder SCC. In some embodiments, the methods provided herein are neoadjuvant therapies and/or adjuvant therapies for treating UC. In some embodiments, the methods provided herein are neoadjuvant therapies and/or adjuvant therapies for treating cervical SCC. In some embodiments, the methods provided herein are neoadjuvant therapies and/or adjuvant therapies for treating penile SCC. In some embodiments, the methods provided herein are neoadjuvant therapies and/or adjuvant therapies for treating vulvar SCC. In some embodiments, the methods provided herein are neoadjuvant therapies and/or adjuvant therapies for treating cutaneous SCC.

In some embodiments, SCC can be advanced SCC, and methods provided herein are neoadjuvant therapies and/or adjuvant therapies for treating advanced SCC. In some embodiments, SCC can be metastatic SCC, and methods provided herein are neoadjuvant therapies and/or adjuvant therapies for treating metastatic SCC. In some embodiments, SCC can be relapsed SCC, and methods provided herein are neoadjuvant therapies and/or adjuvant therapies for treating relapsed SCC. In some embodiments, SCC can be refractory SCC, and methods provided herein are neoadjuvant therapies and/or adjuvant therapies for treating refractory SCC. In some embodiments, the methods provided herein are neoadjuvant therapies and/or adjuvant therapies treating newly diagnosed SCC. In some embodiments, the methods provided herein are neoadjuvant therapies and/or adjuvant therapies treating subjects who have not received any prior treatment for SCC. In some embodiments, the methods provided herein are neoadjuvant therapies and/or adjuvant therapies for HPV negative SCC. In some embodiments, the methods provided herein are neoadjuvant therapies and/or adjuvant therapies for HPV positive SCC.

HNSCC is the 6^{th} most common cancer worldwide, with about 650,000 cases and 200,000 deaths per year worldwide, and about 54,000 new cases per year in the US. It is also the most common cancer in central Asia. HNSCC has 2 different etiologies and corresponding tumor types. The first subtype is associated with tobacco smoking and alcohol consumption, and unrelated to Human papillomavirus (HPV− or HPV negative). The second subtype is associated with infection with high-risk HPV (HPV+ or HPV positive). The second subtype is largely limited to oropharyngeal cancers. HPV+ tumors are distinct entity with better prognosis and may require differential treatments. A significant proportion of HNSCC, particularly oropharyngeal cancers, are caused by HPV infection. High-risk HPV subtype 16 accounts for 85% of all HPV+ tumors in HNSCC. P16 can be used as surrogate marker of HPV infection in HNSCC, particularly in the oropharynx. More accurate HPV testing is available and based on E6/E7 detection (Liang C, et al. Cancer Res. 2012; 72:5004-5013).

HPV+ HNSCC show significantly lower EGFR expression levels than HPV− HNSCC. EGFR amplification only occurs in HPV− HNSCC. High EGFR gene copy number and protein expression are associated with poor clinical outcome in advanced HNSCC.

Currently, first-line therapy for recurrent/metastatic HNSCC include platinum-based doublet (e.g., cisplatin/5-FU or carboplatin/paclitaxel), optionally in combination with anti-EGFR antibody therapy (e.g. cetuximab, panitumumab) or small molecule anti-EGFR inhibitor therapy (e.g. afatinib). Second-line therapy includes taxanes, methotrexate, and/or cetuximab. Anti-EGFR antibody therapy, such as cetuximab (a chimeric IgG1) or panitumumab can be used as a single agent, with chemotherapy (e.g. platinum/5-FU, cisplatin), or with radiation therapy. Despite high EGFR expression levels in HNSCC, single-agent response rate for cetuximab is only 13% with SD rate of 33%, and there is currently no predictive biomarker available to enrich for patients more likely to receive clinical benefit from treatment with cetuximab.

Drugs in development for HNSCC include those targeting PI3K pathway: BKM120 (buparlisib)+cetuximab, BYL719+cetuximab, Temsirolimus+cetuximab, Rigosertib+cetuximab; those targeting MET pathway: Tivantinib+cetuximab, Ficlatuzumab+cetuximab; those targeting EGFR/HER3 pathway Afatinib+cetuximab±paclitaxel, Patritumab; those targeting FGFR pathway: BGJ398; those targeting CDK4/6-cell cycle pathway: Palbociclib, LEE011, abemaciclib, and ribociclib; RTK inhibitor: Anlotinib; AKT inhibitors: MK2206, GSK2110183, and GSK2141795; and chemotherapy: Oral Azacitidine. More recent therapeutic options for HNSCC include immunotherapy, such as anti-PD1 or anti-PDL1 antibodies. Although high cure rates have been achieved for localized and loco-regional disease using surgery, radiation, chemoradiation, and induction chemotherapy, survival rates for recurrent/metastatic diseases remain very poor, and better treatment options are necessary.

In some embodiments, provided herein are methods of neoadjuvant therapies and adjuvant therapies for a subject having an HRas mutant HNSCC, comprising administering a therapeutically effective amount of an FTI to the subject in conjunction with surgery, wherein the HNSCC has an HRas mutant AF that is greater than a reference level. Provided herein are also methods for increasing the responsiveness of an FTI neoadjuvant therapy or adjuvant therapy for HNSCC by selectively treating HNSCC patients having an HRas mutant AF that is greater than a reference level. Provided herein are also methods of predicting responsiveness of a subject having HNSCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having HNSCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapy or adjuvant therapy for a subject having an HRas mutant HNSCC, comprising administering a therapeutically effective amount of an FTI to the subject in conjunction with surgery, wherein the HNSCC has an HRas mutant AF that is greater than 35%. Provided herein are also methods for increasing the responsiveness of an FTI neoadjuvant therapy or adjuvant therapy for HNSCC by selectively treating HNSCC patients having an HRas mutant AF that is greater than 35%. Provided herein are also methods of predicting responsiveness of a subject having HNSCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having HNSCC and administering an FTI neoadjuvant therapy or adjuvant therapy to the subject if the HRas mutant AF is greater than 35%.

In the embodiments, provided herein are neoadjuvant therapies for a subject having an HRas mutant HNSCC, comprising administering a therapeutically effective amount of an FTI to the subject prior to surgery, wherein the HNSCC has an H-Ras mutant AF that is greater than a reference level. In the embodiments, provided herein are adjuvant therapies for a subject having an HRas mutant HNSCC, comprising administering a therapeutically effective amount of an FTI to the subject after surgery, wherein the HNSCC has an H-Ras mutant AF that is greater than a reference level.

In some embodiments, the reference level is 20%, 25%, 30%, 40%, 45%, 50%, 55%, or 60%, and provided herein are methods of neoadjuvant therapy or adjuvant therapy for a subject having an HRas mutant HNSCC in conjunction with surgery, comprising administering a therapeutically effective amount of an FTI to the subject, wherein the HNSCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for HNSCC by selectively treating HNSCC patients having an HRas mutant AF that is greater than 20%, 25%, 30%, 40%, 45%, 50%, 55%, or 60%. Provided herein are also methods of predicting responsiveness of a subject having HNSCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having HNSCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the HNSCC is HNSCC of the trachea. In some embodiments, the HNSCC is HNSCC of the maxilla. In some embodiments, the HNSCC is HNSCC of the oral cavity. In some embodiments, the HNSCC is human papillomavirus (HPV)-negative HNSCC. In some embodiments, the HNSCC is at an advanced stage. In some embodiments, the HNSCC is metastatic HNSCC. In some embodiments, the HNSCC is relapsed HNSCC. In some embodiments, the HNSCC is refractory HNSCC. In some embodiments, the subject having HNSCC is newly diagnosed. In some embodiments, the subject having HNSCC has not received any prior treatment for HNSCC.

SCC of the lung (“LSCC”) accounts for about 30% of all lung cancers. This type of lung cancer tends to be found in the middle of the lungs. Approved treatment options for LSCC include surgery, radiation therapy, chemotherapy, treatment with angiogenesis inhibitors, and immunotherapy. Lung cancer that is only in one lung and that has not spread to other organs is often treated with surgery, if the patient can tolerate it. Radiation therapy can be given as the main treatment in early-stage squamous cell lung cancer if surgery is not possible. In that case, it can be given either with or without chemotherapy. In some cases, radiation therapy is used before or after surgery.

Patients whose lung cancer has spread beyond the lung to local lymph nodes are often given chemotherapy and radiation therapy. Patients with LSCC are often given two chemotherapy agents as first-line therapy. The platinum-based drugs cisplatin or carboplatin are combined with another chemotherapy drug. An example is cisplatin in combination with gemcitabine. The drug, necitumumab (Portrazza™), is also approved by the FDA as first-line treatment of people with metastatic LSCC to be used in combination with cisplatin and gemcitabine. If the LSCC has not been shown to have EGFR mutations, necitumumab seems to work by blocking EGFR protein expression. There are a number of other post-first-line therapy options for LSCC, such as chemotherapy with or without an angiogenesis inhibitor, or immunotherapy, such as nivolumab. The kinase inhibitor afatinib (Gilotrif®), is FDA-approved for the treatment of patients with metastatic LSCC that has progressed after platinum-based chemotherapy. Additional treatment options include ramucirumab (Cyramza®), Nivolumab (Opdivo®), Pembrolizumab (Keytruda) or Atezolizumab (Tecentriq®).

In some embodiments, provided herein are methods of neoadjuvant therapies and adjuvant therapies for a subject having an HRas mutant LSCC, comprising administering a therapeutically effective amount of an FTI to the subject in conjunction with surgery, wherein the LSCC has an HRas mutant AF that is greater than a reference level. Provided herein are also methods for increasing the responsiveness of an FTI neoadjuvant therapy or adjuvant therapy for LSCC by selectively treating LSCC patients having an HRas mutant AF that is greater than a reference level. Provided herein are also methods of predicting responsiveness of a subject having LSCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having LSCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapy or adjuvant therapy for a subject having an HRas mutant LSCC, comprising administering a therapeutically effective amount of an FTI to the subject in conjunction with surgery, wherein the LSCC has an HRas mutant AF that is greater than 35%. Provided herein are also methods for increasing the responsiveness of an FTI neoadjuvant therapy or adjuvant therapy for LSCC by selectively treating LSCC patients having an HRas mutant AF that is greater than 35%. Provided herein are also methods of predicting responsiveness of a subject having LSCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having LSCC and administering an FTI neoadjuvant therapy or adjuvant therapy to the subject if the HRas mutant AF is greater than 35%.

In the embodiments, provided herein are neoadjuvant therapies for a subject having an HRas mutant LSCC, comprising administering a therapeutically effective amount of an FTI to the subject prior to surgery, wherein the LSCC has an H-Ras mutant AF that is greater than a reference level. In the embodiments, provided herein are adjuvant therapies for a subject having an HRas mutant LSCC, comprising administering a therapeutically effective amount of an FTI to the subject after surgery, wherein the LSCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant LSCC in conjunction with surgery, comprising administering a therapeutically effective amount of an FTI to the subject, wherein the LSCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for LSCC by selectively treating LSCC patients having an HRas mutant AF that is greater than 20%, 25%, 30%, 40%, 45%, 50%, 55%, or 60%. Provided herein are also methods of predicting responsiveness of a subject having LSCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having LSCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the LSCC is human papillomavirus (HPV)-negative LSCC. In some embodiments, the LSCC is at an advanced stage. In some embodiments, the LSCC is metastatic LSCC. In some embodiments, the LSCC is relapsed LSCC. In some embodiments, the LSCC is refractory LSCC. In some embodiments, the subject having LSCC is newly diagnosed. In some embodiments, the subject having LSCC has not received any prior treatment for LSCC.

SCC of the thyroid gland (“thyroid SCC” or “TSCC”) can either be a primary or secondary disease, in which it could be due to a direct extension of adjacent lesions or metastasis from other primary foci. The latter are 10-times more common. Primary SCC of the thyroid gland is an unusual type of thyroid malignancy. It is more common in females, with a mean age of occurrence in the sixth decade. Currently, surgical resection of the tumor with adjuvant radiotherapy and chemotherapy is the recommended option. The extent of the surgical resection is poorly defined. However, in advanced stage diseases, the extensive and invasive nature of the thyroid SCC can be the main factor of surgical failure. Moreover, primary thyroid SCC is also relatively resistant to radiotherapy, while standard chemotherapy has shown minimal to absent response towards the disease. General prognosis of primary SCC of the thyroid is very unfavourable regardless of the treatment, due to its aggressive nature. Better treatment options are needed.

In some embodiments, provided herein are methods of neoadjuvant therapy and adjuvant therapy for a subject having an HRas mutant thyroid SCC, comprising administering a therapeutically effective amount of an FTI to the subject in conjunction with surgery, wherein the thyroid SCC has an HRas mutant AF that is greater than a reference level. Provided herein are also methods for increasing the responsiveness of an FTI neoadjuvant therapy or adjuvant therapy for thyroid SCC by selectively treating thyroid SCC patients having an HRas mutant AF that is greater than a reference level. Provided herein are also methods of predicting responsiveness of a subject having thyroid SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having thyroid SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant thyroid SCC, comprising administering a therapeutically effective amount of an FTI to the subject in conjunction with surgery, wherein the thyroid SCC has an HRas mutant AF that is greater than 35%. Provided herein are also methods for increasing the responsiveness of an FTI neoadjuvant therapy or adjuvant therapy for thyroid SCC by selectively treating thyroid SCC patients having an HRas mutant AF that is greater than 35%. Provided herein are also methods of predicting responsiveness of a subject having thyroid SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having thyroid SCC and administering an FTI neoadjuvant therapy or adjuvant therapy to the subject if the HRas mutant AF is greater than 35%.

In the embodiments, provided herein are neoadjuvant therapies for a subject having an HRas mutant thyroid SCC, comprising administering a therapeutically effective amount of an FTI to the subject prior to surgery, wherein the thyroid SCC has an H-Ras mutant AF that is greater than a reference level. In the embodiments, provided herein are adjuvant therapies for a subject having an HRas mutant thyroid SCC, comprising administering a therapeutically effective amount of an FTI to the subject after surgery, wherein the thyroid SCC has an H-Ras mutant AF that is greater than a reference level.

In some embodiments, the reference level is 20%, 25%, 30%, 40%, 45%, 50%, 55%, or 60%, and provided herein are methods of neoadjuvant therapy or adjuvant therapy for a subject having an HRas mutant thyroid SCC in conjunction with surgery, comprising administering a therapeutically effective amount of an FTI to the subject, wherein the thyroid SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for thyroid SCC by selectively treating thyroid SCC patients having an HRas mutant AF that is greater than 20%, 25%, 30%, 40%, 45%, 50%, 55%, or 60%. Provided herein are also methods of predicting responsiveness of a subject having thyroid SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having thyroid SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the thyroid SCC is human papillomavirus (HPV)-negative thyroid SCC. In some embodiments, the thyroid SCC is at an advanced stage. In some embodiments, the thyroid SCC is metastatic thyroid SCC. In some embodiments, the thyroid SCC is relapsed thyroid SCC. In some embodiments, the thyroid SCC is refractory thyroid SCC. In some embodiments, the subject having thyroid SCC is newly diagnosed. In some embodiments, the subject having thyroid SCC has not received any prior treatment for thyroid SCC.

Esophageal squamous cell carcinoma (“Esophageal SCC” or “ESCC”) is one of the most aggressive squamous cell carcinomas and is highly prevalent in Asia. Patients with ESCC are treated endoscopically or with surgery, chemotherapy, or radiotherapy, based on tumor stage. Minimally invasive treatments help improve the quality of life of patients who undergo such treatments. Early-stage ESCC, with negligible risk of metastasis to the lymph node, can be cured by endoscopic local treatment, such as ER and/or an ablative method (e.g., radiofrequency ablation or photodynamic therapy). Surgery is also used widely to obtain locoregional control and has an important role in the treatment of esophageal cancer. Neoadjuvant or neoadjuvant chemoradiation is performed as standard treatment for locally advanced ESCC. Combinations of cisplatin and 5-FU are commonly used in chemotherapy for patients with unresectable locally advanced or metastatic ESCC, which is believed to be better than the best supportive care. Target therapies such as anti-EGFR antibodies (e.g. cetuximab), anti-PD1/PD-L1 antibodies are also under investigation.

In some embodiments, provided herein are methods of neoadjuvant therapies and adjuvant therapies for a subject having an HRas mutant esophageal SCC, comprising administering a therapeutically effective amount of an FTI to the subject in conjunction with surgery, wherein the esophageal SCC has an HRas mutant AF that is greater than a reference level. Provided herein are also methods for increasing the responsiveness of an FTI neoadjuvant therapy or adjuvant therapy for esophageal SCC by selectively treating esophageal SCC patients having an HRas mutant AF that is greater than a reference level. Provided herein are also methods of predicting responsiveness of a subject having esophageal SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having esophageal SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant esophageal SCC, comprising administering a therapeutically effective amount of an FTI to the subject in conjunction with surgery, wherein the esophageal SCC has an HRas mutant AF that is greater than 35%. Provided herein are also methods for increasing the responsiveness of an FTI neoadjuvant therapy or adjuvant therapy for esophageal SCC by selectively treating esophageal SCC patients having an HRas mutant AF that is greater than 35%. Provided herein are also methods of predicting responsiveness of a subject having esophageal SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having esophageal SCC and administering an FTI neoadjuvant therapy or adjuvant therapy to the subject if the HRas mutant AF is greater than 35%.

In the embodiments, provided herein are neoadjuvant therapies for a subject having an HRas mutant esophageal SCC, comprising administering a therapeutically effective amount of an FTI to the subject prior to surgery, wherein the esophageal SCC has an H-Ras mutant AF that is greater than a reference level. In the embodiments, provided herein are adjuvant therapies for a subject having an HRas mutant esophageal SCC, comprising administering a therapeutically effective amount of an FTI to the subject after surgery, wherein the esophageal SCC has an H-Ras mutant AF that is greater than a reference level.

In some embodiments, the reference level is 20%, 25%, 30%, 40%, 45%, 50%, 55%, or 60%, and provided herein are methods of neoadjuvant therapy or adjuvant therapy for a subject having an HRas mutant esophageal SCC in conjunction with surgery, comprising administering a therapeutically effective amount of an FTI to the subject, wherein the esophageal SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for esophageal SCC by selectively treating esophageal SCC patients having an HRas mutant AF that is greater than 20%, 25%, 30%, 40%, 45%, 50%, 55%, or 60%. Provided herein are also methods of predicting responsiveness of a subject having esophageal SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having esophageal SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the esophageal SCC is human papillomavirus (HPV)-negative esophageal SCC. In some embodiments, the esophageal SCC is at an advanced stage. In some embodiments, the esophageal SCC is metastatic esophageal SCC. In some embodiments, the esophageal SCC is relapsed esophageal SCC. In some embodiments, the esophageal SCC is refractory esophageal SCC. In some embodiments, the subject having esophageal SCC is newly diagnosed. In some embodiments, the subject having esophageal SCC has not received any prior treatment for esophageal SCC.

Bladder squamous cell carcinoma (“Bladder SCC” or “BSCC”) usually presents at a late stage and portends poor prognosis. Bladder SCC represent 2-5% of bladder malignancies in the U.S. BSCC is divided into two subtypes, BSCC associated with bilharzia infection (schistosomiasis), i.e. bilharzial-associated BSCC (B-BSCC) and BSCC not associated with bilharziasis, i.e. non-bilharzial-associated SCC (NB-BSCC). B-BSCC and NB-BSCC differ in their epidemiology, natural history, and clinicopathological features. B-BSCC is predominantly found in regions where schistosomiasis is endemic, such as in the Middle East, Southeast Asia, and South America. In the USA, NB-BSCC has been reported in patients with spinal cord injury (SCI), particularly following long-term use of an indwelling catheter. Patients with NB-BSCC are generally diagnosed at a late stage and present with poor prognosis. Both B-BSCC and NB-BSCC are treated with radical cystectomy (RC); the use of other treatments, including neoadjuvant and adjuvant therapies in conjunction with RC, is not well established. Additional studies incorporating multimodal approaches, contemporary radiation techniques, immunotherapies and systemic therapies are also needed.

In some embodiments, provided herein are methods of neoadjuvant therapies and adjuvant therapies for a subject having an HRas mutant bladder SCC, comprising administering a therapeutically effective amount of an FTI to the subject in conjunction with surgery, wherein the bladder SCC has an HRas mutant AF that is greater than a reference level. Provided herein are also methods for increasing the responsiveness of an FTI neoadjuvant therapy or adjuvant therapy for bladder SCC by selectively treating bladder SCC patients having an HRas mutant AF that is greater than a reference level. Provided herein are also methods of predicting responsiveness of a subject having bladder SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having bladder SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant bladder SCC, comprising administering a therapeutically effective amount of an FTI to the subject in conjunction with surgery, wherein the bladder SCC has an HRas mutant AF that is greater than 35%. Provided herein are also methods for increasing the responsiveness of an FTI neoadjuvant therapy or adjuvant therapy for bladder SCC by selectively treating bladder SCC patients having an HRas mutant AF that is greater than 35%. Provided herein are also methods of predicting responsiveness of a subject having bladder SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having bladder SCC and administering an FTI neoadjuvant therapy or adjuvant therapy to the subject if the HRas mutant AF is greater than 35%.

In the embodiments, provided herein are neoadjuvant therapies for a subject having an HRas mutant bladder SCC, comprising administering a therapeutically effective amount of an FTI to the subject prior to surgery, wherein the bladder SCC has an H-Ras mutant AF that is greater than a reference level. In the embodiments, provided herein are adjuvant therapies for a subject having an HRas mutant bladder SCC, comprising administering a therapeutically effective amount of an FTI to the subject after surgery, wherein the bladder SCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant bladder SCC in conjunction with surgery, comprising administering a therapeutically effective amount of an FTI to the subject, wherein the bladder SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for bladder SCC by selectively treating bladder SCC patients having an HRas mutant AF that is greater than 20%, 25%, 30%, 40%, 45%, 50%, 55%, or 60%. Provided herein are also methods of predicting responsiveness of a subject having bladder SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having bladder SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the bladder SCC is human papillomavirus (HPV)-negative bladder SCC. In some embodiments, the bladder SCC is at an advanced stage. In some embodiments, the bladder SCC is metastatic bladder SCC. In some embodiments, the bladder SCC is relapsed bladder SCC. In some embodiments, the bladder SCC is refractory bladder SCC. In some embodiments, the subject having bladder SCC is newly diagnosed. In some embodiments, the subject having bladder SCC has not received any prior treatment for bladder SCC.

Urothelial carcinoma (UC) is an indication with a 5-year survival rate of 77%. Cells of UC commonly exhibit squamous differentiation and characteristics, defined by the presence of intercellular bridges, keratinization, or both. Liu et al., Cancer Control 24(1):78-82 (2017).

In some embodiments, provided herein are methods of neoadjuvant therapies and adjuvant therapies for a subject having an HRas mutant UC, comprising administering a therapeutically effective amount of an FTI to the subject in conjunction with surgery, wherein the UC has an HRas mutant AF that is greater than a reference level. Provided herein are also methods for increasing the responsiveness of an FTI neoadjuvant therapy or adjuvant therapy for UC by selectively treating UC patients having an HRas mutant AF that is greater than a reference level. Provided herein are also methods of predicting responsiveness of a subject having UC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having UC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant UC, comprising administering a therapeutically effective amount of an FTI to the subject in conjunction with surgery, wherein the UC has an HRas mutant AF that is greater than 35%. Provided herein are also methods for increasing the responsiveness of an FTI neoadjuvant therapy or adjuvant therapy for UC by selectively treating UC patients having an HRas mutant AF that is greater than 35%. Provided herein are also methods of predicting responsiveness of a subject having UC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having UC and administering an FTI neoadjuvant therapy or adjuvant therapy to the subject if the HRas mutant AF is greater than 35%.

In the embodiments, provided herein are neoadjuvant therapies for a subject having an HRas mutant UC, comprising administering a therapeutically effective amount of an FTI to the subject prior to surgery, wherein the UC has an H-Ras mutant AF that is greater than a reference level. In the embodiments, provided herein are adjuvant therapies for a subject having an HRas mutant UC, comprising administering a therapeutically effective amount of an FTI to the subject after surgery, wherein the UC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant UC in conjunction with surgery, comprising administering a therapeutically effective amount of an FTI to the subject, wherein the UC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for UC by selectively treating UC patients having an HRas mutant AF that is greater than 20%, 25%, 30%, 40%, 45%, 50%, 55%, or 60%. Provided herein are also methods of predicting responsiveness of a subject having UC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having UC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the UC is human papillomavirus (HPV)-negative UC. In some embodiments, the UC is at an advanced stage. In some embodiments, the UC is metastatic UC. In some embodiments, the UC is relapsed UC. In some embodiments, the UC is refractory UC. In some embodiments, the subject having UC is newly diagnosed. In some embodiments, the subject having UC has not received any prior treatment for UC.

Cutaneous SCC is the second most common form of skin cancer. More than 1 million cases of squamous cell carcinoma are diagnosed each year in the U.S. Incidence has increased up to 200 percent in the past three decades in the U.S., and more than 15,000 Americans die each year from the disease. Cumulative, long-term exposure to ultraviolet (UV) radiation from the sun over your lifetime causes most SCCs. If untreated, cutaneous SCC can metastasize to local lymph nodes, distant tissues and organs and can become life-threatening. Treatment options include Mohs surgery, excisional surgery, curettage and electrodesiccation, cryosurgery, laser surgery, radiation therapy, photodynamic therapy (PDT), and topical medicines (e.g. 5-fluorouracil (5-FU) and imiquimod).

In some embodiments, provided herein are methods of neoadjuvant therapies and adjuvant therapies for a subject having an HRas mutant cutaneous SCC, comprising administering a therapeutically effective amount of an FTI to the subject in conjunction with surgery, wherein the cutaneous SCC has an HRas mutant AF that is greater than a reference level. Provided herein are also methods for increasing the responsiveness of an FTI neoadjuvant therapy or adjuvant therapy for cutaneous SCC by selectively treating cutaneous SCC patients having an HRas mutant AF that is greater than a reference level. Provided herein are also methods of predicting responsiveness of a subject having cutaneous SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having cutaneous SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant cutaneous SCC, comprising administering a therapeutically effective amount of an FTI to the subject in conjunction with surgery, wherein the cutaneous SCC has an HRas mutant AF that is greater than 35%. Provided herein are also methods for increasing the responsiveness of an FTI neoadjuvant therapy or adjuvant therapy for cutaneous SCC by selectively treating cutaneous SCC patients having an HRas mutant AF that is greater than 35%. Provided herein are also methods of predicting responsiveness of a subject having cutaneous SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having cutaneous SCC and administering an FTI neoadjuvant therapy or adjuvant therapy to the subject if the HRas mutant AF is greater than 35%.

In the embodiments, provided herein are neoadjuvant therapies for a subject having an HRas mutant cutaneous SCC, comprising administering a therapeutically effective amount of an FTI to the subject prior to surgery, wherein the cutaneous SCC has an H-Ras mutant AF that is greater than a reference level. In the embodiments, provided herein are adjuvant therapies for a subject having an HRas mutant cutaneous SCC, comprising administering a therapeutically effective amount of an FTI to the subject after surgery, wherein the cutaneous SCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant cutaneous SCC in conjunction with surgery, comprising administering a therapeutically effective amount of an FTI to the subject, wherein the cutaneous SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for cutaneous SCC by selectively treating cutaneous SCC patients having an HRas mutant AF that is greater than 20%, 25%, 30%, 40%, 45%, 50%, 55%, or 60%. Provided herein are also methods of predicting responsiveness of a subject having cutaneous SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having cutaneous SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the cutaneous SCC is human papillomavirus (HPV)-negative cutaneous SCC. In some embodiments, the cutaneous SCC is at an advanced stage. In some embodiments, the cutaneous SCC is metastatic cutaneous SCC. In some embodiments, the cutaneous SCC is relapsed cutaneous SCC. In some embodiments, the cutaneous SCC is refractory cutaneous SCC. In some embodiments, the subject having cutaneous SCC is newly diagnosed. In some embodiments, the subject having cutaneous SCC has not received any prior treatment for cutaneous SCC.

Cervical cancer is a malignancy of the cervix, the 2nd or 3rd most common cancer in women worldwide. Over half a million cases are diagnosed each year. Between 80% and 90% of cervical cancer cases involve the squamous cells (cervical SCCs, also referred to as SCC of uterine cervix). The rest start from glandular cells and are adenocarcinoma. Depending on the stage of the cervical SCC, treatment options include cryosurgery, laser surgery, Loop electrosurgical excision procedure (LEEP/LEETZ), hysterectomy, radical trachelectomy, chemotherapy (e.g. cisplatin or cisplatin plus fluorouracil), and radiation therapy.

In some embodiments, provided herein are methods of neoadjuvant therapies and adjuvant therapies for a subject having an HRas mutant cervical SCC, comprising administering a therapeutically effective amount of an FTI to the subject in conjunction with surgery, wherein the cervical SCC has an HRas mutant AF that is greater than a reference level. Provided herein are also methods for increasing the responsiveness of an FTI neoadjuvant therapy or adjuvant therapy for cervical SCC by selectively treating cervical SCC patients having an HRas mutant AF that is greater than a reference level. Provided herein are also methods of predicting responsiveness of a subject having cervical SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having cervical SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant cervical SCC, comprising administering a therapeutically effective amount of an FTI to the subject in conjunction with surgery, wherein the cervical SCC has an HRas mutant AF that is greater than 35%. Provided herein are also methods for increasing the responsiveness of an FTI neoadjuvant therapy or adjuvant therapy for cervical SCC by selectively treating cervical SCC patients having an HRas mutant AF that is greater than 35%. Provided herein are also methods of predicting responsiveness of a subject having cervical SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having cervical SCC and administering an FTI neoadjuvant therapy or adjuvant therapy to the subject if the HRas mutant AF is greater than 35%.

In the embodiments, provided herein are neoadjuvant therapies for a subject having an HRas mutant cervical SCC, comprising administering a therapeutically effective amount of an FTI to the subject prior to surgery, wherein the cervical SCC has an H-Ras mutant AF that is greater than a reference level. In the embodiments, provided herein are adjuvant therapies for a subject having an HRas mutant cervical SCC, comprising administering a therapeutically effective amount of an FTI to the subject after surgery, wherein the cervical SCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant cervical SCC in conjunction with surgery, comprising administering a therapeutically effective amount of an FTI to the subject, wherein the cervical SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for cervical SCC by selectively treating cervical SCC patients having an HRas mutant AF that is greater than 20%, 25%, 30%, 40%, 45%, 50%, 55%, or 60%. Provided herein are also methods of predicting responsiveness of a subject having cervical SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having cervical SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the cervical SCC is human papillomavirus (HPV)-negative cervical SCC. In some embodiments, the cervical SCC is at an advanced stage. In some embodiments, the cervical SCC is metastatic cervical SCC. In some embodiments, the cervical SCC is relapsed cervical SCC. In some embodiments, the cervical SCC is refractory cervical SCC. In some embodiments, the subject having cervical SCC is newly diagnosed. In some embodiments, the subject having cervical SCC has not received any prior treatment for cervical SCC.

Vulvar cancer is the fourth most common gynecologic cancer in high-resource countries and comprises approximately 5 to 6 percent of malignancies of the female genital tract. Although various histologic subtypes of vulvar cancer exist, most cancers of the vulva are SCCs. In the United States, vulvar cancer accounts for nearly 6% of cancers of the female reproductive organs and 0.7% of all cancers in women. Depending on the stage, treatment options for vulvar SCC include surgery, local excision, radical vulvectomy, radiation therapy, chemotherapy, pelvic exenteration.

In some embodiments, provided herein are methods of neoadjuvant therapies and adjuvant therapies for a subject having an HRas mutant vulvar SCC, comprising administering a therapeutically effective amount of an FTI to the subject in conjunction with surgery, wherein the vulvar SCC has an HRas mutant AF that is greater than a reference level. Provided herein are also methods for increasing the responsiveness of an FTI neoadjuvant therapy or adjuvant therapy for vulvar SCC by selectively treating vulvar SCC patients having an HRas mutant AF that is greater than a reference level. Provided herein are also methods of predicting responsiveness of a subject having vulvar SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having vulvar SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant vulvar SCC, comprising administering a therapeutically effective amount of an FTI to the subject in conjunction with surgery, wherein the vulvar SCC has an HRas mutant AF that is greater than 35%. Provided herein are also methods for increasing the responsiveness of an FTI neoadjuvant therapy or adjuvant therapy for vulvar SCC by selectively treating vulvar SCC patients having an HRas mutant AF that is greater than 35%. Provided herein are also methods of predicting responsiveness of a subject having vulvar SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having vulvar SCC and administering an FTI neoadjuvant therapy or adjuvant therapy to the subject if the HRas mutant AF is greater than 35%.

In the embodiments, provided herein are neoadjuvant therapies for a subject having an HRas mutant vulvar SCC, comprising administering a therapeutically effective amount of an FTI to the subject prior to surgery, wherein the vulvar SCC has an H-Ras mutant AF that is greater than a reference level. In the embodiments, provided herein are adjuvant therapies for a subject having an HRas mutant vulvar SCC, comprising administering a therapeutically effective amount of an FTI to the subject after surgery, wherein the vulvar SCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant vulvar SCC in conjunction with surgery, comprising administering a therapeutically effective amount of an FTI to the subject, wherein the vulvar SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for vulvar SCC by selectively treating vulvar SCC patients having an HRas mutant AF that is greater than 20%, 25%, 30%, 40%, 45%, 50%, 55%, or 60%. Provided herein are also methods of predicting responsiveness of a subject having vulvar SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having vulvar SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the vulvar SCC is human papillomavirus (HPV)-negative vulvar SCC. In some embodiments, the vulvar SCC is at an advanced stage. In some embodiments, the vulvar SCC is metastatic vulvar SCC. In some embodiments, the vulvar SCC is relapsed vulvar SCC. In some embodiments, the vulvar SCC is refractory vulvar SCC. In some embodiments, the subject having vulvar SCC is newly diagnosed. In some embodiments, the subject having vulvar SCC has not received any prior treatment for vulvar SCC.

Penile cancer is a rare type of cancer that commonly affects men over the age of 50. Penile SCC accounts for more than 90% of cases. Depending on the stage, treatment options include surgery, radiotherapy and chemotherapy.

In some embodiments, provided herein are methods of neoadjuvant therapies and adjuvant therapies for a subject having an HRas mutant penile SCC, comprising administering a therapeutically effective amount of an FTI to the subject in conjunction with surgery, wherein the penile SCC has an HRas mutant AF that is greater than a reference level. Provided herein are also methods for increasing the responsiveness of an FTI neoadjuvant therapy or adjuvant therapy for penile SCC by selectively treating penile SCC patients having an HRas mutant AF that is greater than a reference level. Provided herein are also methods of predicting responsiveness of a subject having penile SCC to an FTI neoadjuvant therapy or adjuvant therapy based on the HRas mutant AF, wherein a subject is predicted to be likely responsive if the subject has HRas mutant AF that is greater than a reference level. In some embodiments, methods provided herein also include measuring HRas mutant AF in a sample of a subject having penile SCC and administering an FTI neoadjuvant therapy or adjuvant therapy to the subject if the HRas mutant AF is greater than the reference level. The FTI can be any FTI known in the art, including those described herein. For example, the FTI can be tipifarnib, lonafarnib, arglabin, perrilyl alcohol, L778123, L739749, FTI-277, L744832, CP-609,754, R208176, AZD3409, or BMS-214662. In some embodiments, the FTI is tipifarnib. In some embodiments, the methods include analyzing a sample from the subject to measure the HRas mutant AF in the sample.

In some embodiments, the reference level is 35%, and provided herein are methods of neoadjuvant therapies or adjuvant therapies for a subject having an HRas mutant penile SCC, comprising administering a therapeutically effective amount of an FTI to the subject in conjunction with surgery, wherein the penile SCC has an HRas mutant AF that is greater than 35%. Provided herein are also methods for increasing the responsiveness of an FTI neoadjuvant therapy or adjuvant therapy for penile SCC by selectively treating penile SCC patients having an HRas mutant AF that is greater than 35%. Provided herein are also methods of predicting responsiveness of a subject having penile SCC to an FTI neoadjuvant therapy or adjuvant therapy based on the HRas mutant AF, wherein a subject is predicted to be likely responsive if the subject has HRas mutant AF that is greater than 35%. In some embodiments, methods provided herein also include measuring HRas mutant AF in a sample of a subject having penile SCC and administering an FTI neoadjuvant therapy or adjuvant therapy to the subject if the HRas mutant AF is greater than 35%.

In the embodiments, provided herein are neoadjuvant therapies for a subject having an HRas mutant penile SCC, comprising administering a therapeutically effective amount of an FTI to the subject prior to surgery, wherein the penile SCC has an H-Ras mutant AF that is greater than a reference level. In the embodiments, provided herein are adjuvant therapies for a subject having an HRas mutant penile SCC, comprising administering a therapeutically effective amount of an FTI to the subject after surgery, wherein the penile SCC has an H-Ras mutant AF that is greater than a reference level.

In some embodiments, the reference level is 20%, 25%, 30%, 40%, 45%, 50%, 55%, or 60%, and provided herein are methods of neoadjuvant therapies or adjuvant therapies for a subject having an HRas mutant penile SCC in conjunction with surgery, comprising administering a therapeutically effective amount of an FTI to the subject, wherein the penile SCC has an HRas mutant AF that is greater than 20%, 25%, 30%, 40%, 45%, 50%, 55%, or 60%. Provided herein are also methods for increasing the responsiveness of an FTI neoadjuvant therapy or adjuvant therapy for penile SCC by selectively treating penile SCC patients having an HRas mutant AF that is greater than 20%, 25%, 30%, 40%, 45%, 50%, 55%, or 60%. Provided herein are also methods of predicting responsiveness of a subject having penile SCC to an FTI neoadjuvant therapy or adjuvant therapy based on the HRas mutant AF, wherein a subject is predicted to be likely responsive if the subject has HRas mutant AF that is greater than 20%, 25%, 30%, 40%, 45%, 50%, 55%, or 60%. In some embodiments, methods provided herein also include measuring HRas mutant AF in a sample of a subject having penile SCC and administering an FTI neoadjuvant therapy or adjuvant therapy to the subject if the HRas mutant AF is greater than 20%, 25%, 30%, 40%, 45%, 50%, 55%, or 60%. In some embodiments, the AF reference level can be 21%, 22%, 23%, 24%, 25%, 26%, 27%, 28%, 29%, 30%, 31%, 32%, 33%, 34%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the penile SCC is human papillomavirus (HPV)-negative penile SCC. In some embodiments, the penile SCC is at an advanced stage. In some embodiments, the penile SCC is metastatic penile SCC. In some embodiments, the penile SCC is relapsed penile SCC. In some embodiments, the penile SCC is refractory penile SCC. In some embodiments, the subject having penile SCC is newly diagnosed. In some embodiments, the subject having penile SCC has not received any prior treatment for penile SCC.

In some embodiments, the methods provided herein also include obtaining a sample from the subject. The sample used in the methods provided herein includes body fluids from a subject or a tumour biopsy from the subject.

In some embodiments, the sample used in the present methods includes a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy. In some embodiments, the sample used in the present methods includes an aspirate (e.g., bone marrow aspirate). In some embodiments, the sample is a lymph node biopsy. In some embodiments, the sample can be a frozen tissue sample. In some embodiments, the sample can be a formalin-fixed paraffin-embedded (“FFPE”) tissue sample. In some embodiments, the sample can be a deparaffinised tissue section. In some embodiments, the sample can be a liver sample. In some embodiments, the sample can be a testicle sample. In some embodiments, the sample can be a spleen sample. In some embodiments, the sample can be a lymph node sample.

In some embodiments, the sample is a body fluid sample. Non-limiting examples of body fluids include blood (e.g., peripheral whole blood, peripheral blood), blood plasma, bone marrow, amniotic fluid, aqueous humor, bile, lymph, menses, serum, urine, cerebrospinal fluid surrounding the brain and the spinal cord, synovial fluid surrounding bone joints. In some embodiments, the sample can be a spinal fluid sample.

In some embodiments, the sample is a blood sample. The blood sample can contain tumor DNA. The blood sample can be a whole blood sample, a partially purified blood sample, or a peripheral blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, NK cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.). In some embodiments, the sample is serum. In some embodiments, the sample is plasma. In one embodiment, the sample is a bone marrow sample.

In certain embodiments, the sample used in the methods provided herein includes a plurality of cells. Such cells can include any type of cells, e.g., stem cells, blood cells (e.g., PBMCs), lymphocytes, NK cells, B cells, T cells, monocytes, granulocytes, immune cells, or tumor or cancer cells. Specific cell populations can be obtained using a combination of commercially available antibodies (e.g., Quest Diagnostic (San Juan Capistrano, Calif.); Dako (Denmark)). In some embodiments, the sample is isolated cells.

In certain embodiments, the sample used in the methods provided herein includes a plurality of cells from the diseased tissue, e.g., a tumor sample from the subject having SCC. In some embodiments, the cells can be obtained from the tumor tissue, such as a tumor biopsy or a tumor explants. In certain embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸. Different types of procedures are available to obtain a tumor biopsy from a patient, including skin biopsy, shave (tangential) biopsy, punch biopsy, incisional biopsy (which removes a portion of the tumor) and excisional biopsy (which removes the entire tumor). Lymph node biopsies are usually performed to examiner whether cancer has spread. Both fine needle aspiration (FNA) biopsy and surgical (excisional) lymph node biopsy are available options. The FNA biopsy allows the patient to use a thin needle to obtain a small fragment of the lymph node, which is less invasive than the surgical option, but may not always provide a large enough sample to find cancer cells.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

Methods for measuring HRas mutant AF are well known in the art. In some embodiments, the methods include sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay. In some embodiments, the HRas mutant AF gene is determined using standard sequencing methods, including, for example, Sanger sequencing, next generation sequencing (NGS). In some embodiments, the HRas mutant AF can be determined an NGS-based assay. In some embodiments, the HRas mutant AF can be determined by a qualitative PCR-based assay. In some embodiments, the HRas mutant AF is determined using MS.

In some embodiments, methods provided herein include amplifying HRas nucleic acid from the patient's tumor sample and sequencing the amplified nucleic acid. HRas nucleic acid can be amplified using methods known in the art. In some embodiments, nucleic acid can be obtained from the patient's tumor sample by any method known to the person skilled in the art. For example, any commercial kit may be used to isolate the genomic DNA, or mRNA from a tumor sample, such as e.g. the Qlamp DNA mini kit, or RNeasy mini kit (Qiagen, Hilden, Germany). For example, if mRNA was isolated from the patient's tumor sample, cDNA synthesis can be carried out prior to the methods as disclosed herein, according to any known technology in the art.

For example, the nucleic acid to be isolated from a tumor can for example be one of genomic DNA, total RNA, mRNA or poly(A)+mRNA. For example, if mRNA has been isolated from the the patient's tumor sample, the mRNA (total mRNA or poly(A)+mRNA) may be used for cDNA synthesis according to well established technologies in prior art, such as those provided in commercial cDNA synthesis kits, e.g. Superscript® III First Strand Synthesis Kit. The cDNA can then be further amplified by means of e.g. PCR and subsequently subjected to sequencing by e.g. Sanger sequencing or pyro-sequencing to determine the nucleotide sequence of e.g. codons 12 and 13 of the RAS gene, e.g. H-RAS, N-RAS or KRAS. Alternatively, the PCR product can e.g. also be subcloned into a TA TOPO cloning vector for sequencing. Other technologies than sequencing to determine HRas mutant AF can be used in the methods provided herein such as e.g. Single Nucleotide Primer Extension (SNPE) (PLoS One. 2013 Aug. 21; 8(8):e72239); DNA microarray, Mass Spectrometry (MS) (e.g. matrix-assisted laser desorption/ionization time-of-flight (MALDI-TOF) mass spectrometry), Single Nucleotide Polymorphism (SNP), denaturing high-performance liquid chromatography (DHPLC), or Restriction Fragment Length Polymorphism (RFLP) assay.

For example, Single Nucleotide Polymorphism (SNP) Assay can be used for determining the HRas mutant AF in a sample. The SNP assay can be performed on the HT7900 from Applied Biosystems, following the allelic discrimination assay protocol provided by the manufacturer. HRas mutant AF can also be determined by DHPLC or RFLP, or any other methods known in the art. Bowen et al., Blood, 106:2113-2119 (2005); Bowen et al., Blood, 101:2770-2774 (2003); Nishikawa et al., Clin Chim Acta., 318:107-112 (2002); Lin S Y et al., Am J Clin Pathol. 100:686-689 (1993); O'Leary J J et al., J Clin Pathol. 51:576-582 (1998).

As a person of ordinary skill in the art would understand, any methods described herein or otherwise known in the art for analyzing Ras mutation can be used to determining the presence or absence of a HRas mutation.

### B. Pharmaceutical Compositions

In some embodiments, provided herein is a method of neoadjuvant therapy or adjuvant therapy for a subject having an HRas mutant SCC comprising administering a subject with an FTI or a pharmaceutical composition having an FTI. The pharmaceutical compositions provided herein contain therapeutically effective amounts of an FTI and a pharmaceutically acceptable carrier, diluent or excipient. In some embodiments, the FTI is tipifarnib; lonafarnib (also known as SCH-66336); arglabin; perrilyl alcohol; CP-609,754, BMS 214662; L778123; L744832; L739749; R208176; AZD3409; or FTI-277. In some embodiments, the FTI is tipifarnib.

The FTI can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the FTI is formulated into pharmaceutical compositions using techniques and procedures well known in the art (see, e.g., Ansel Introduction to Pharmaceutical Dosage Forms, Seventh Edition 1999).

In the compositions, effective concentrations of the FTI and pharmaceutically acceptable salts is (are) mixed with a suitable pharmaceutical carrier or vehicle. In certain embodiments, the concentrations of the FTI in the compositions are effective for delivery of an amount, upon administration, that treats, prevents, or ameliorates one or more of the symptoms and/or progression of cancer, including haematological cancers and solid tumors.

The compositions can be formulated for single dosage administration. To formulate a composition, the weight fraction of the FTI is dissolved, suspended, dispersed or otherwise mixed in a selected vehicle at an effective concentration such that the treated condition is relieved or ameliorated. Pharmaceutical carriers or vehicles suitable for administration of the FTI provided herein include any such carriers known to those skilled in the art to be suitable for the particular mode of administration.

In addition, the FTI can be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of an FTI provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The FTI is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of FTI in the pharmaceutical composition will depend on absorption, tissue distribution, inactivation and excretion rates of the FTI, the physicochemical characteristics of the FTI, the dosage schedule, and amount administered as well as other factors known to those of skill in the art. For example, the amount that is delivered is sufficient to ameliorate one or more of the symptoms of cancer, including hematopoietic cancers and solid tumors.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The FTI may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the FTI exhibits insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from nontoxic carrier may be prepared. For oral administration, a pharmaceutically acceptable nontoxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1-85% or about 75-95%.

The FTI or pharmaceutically acceptable salts can be prepared with carriers that protect the compound against rapid elimination from the body, such as time release formulations or coatings.

The compositions can include other active compounds to obtain desired combinations of properties. The compounds provided herein, or pharmaceutically acceptable salts thereof as described herein, can also be administered together with another pharmacological agent known in the general art to be of value in treating one or more of the diseases or medical conditions referred to hereinabove, such as diseases related to oxidative stress.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in noneffervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also provided herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the FTI is adjusted so that an injection provides an effective amount to produce the desired pharmacological effect. The exact dose depends on the age, weight and condition of the patient or animal as is known in the art. The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an FTI is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FTI can be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They can also be reconstituted and formulated as solids or gels.

The sterile, lyophilized powder is prepared by dissolving an FTI provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FTI or pharmaceutical composition having an FTI can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The FTI or pharmaceutical composition having an FTI can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, may be formulated as 0.01%-10% isotonic solutions, pH about 5-7, with appropriate salts.

Other routes of administration, such as transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono , di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams. Tablets and capsules for rectal administration are manufactured using the same pharmaceutically acceptable substance and by the same methods as for formulations for oral administration.

The FTI or pharmaceutical composition having an FTI provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of FTI using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the FTI can be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The F can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

The FTI or pharmaceutical composition of FTI can be packaged as articles of manufacture containing packaging material, a compound or pharmaceutically acceptable salt thereof provided herein, which is used for treatment, prevention or amelioration of one or more symptoms or progression of cancer, including haematological cancers and solid tumors, and a label that indicates that the compound or pharmaceutically acceptable salt thereof is used for treatment, prevention or amelioration of one or more symptoms or progression of cancer, including haematological cancers and solid tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In some embodiments, a therapeutically effective amount of the pharmaceutical composition having an FTI is administered orally or parenterally.

In some embodiments, the FTI is administered at a daily dose of from 0.05 up to 1800 mg/kg. In some embodiments, the FTI is administered at a daily dose of from 0.05 up to 1500 mg/kg. In some embodiments, the FTI is administered at a daily dose of from 0.05 up to 500 mg/kg. In some embodiments, the FTI is administered in an amount of 0.05 mg/kg daily, 0.1 mg/kg daily, 0.2 mg/kg daily, 0.5 mg/kg daily, 1 mg/kg daily, 2 mg/kg daily, 5 mg/kg daily, 10 mg/kg daily, 20 mg/kg daily, 50 mg/kg daily, 100 mg/kg daily, 200 mg/kg daily, 300 mg/kg daily, 400 mg/kg daily, 500 mg/kg daily, 600 mg/kg daily, 700 mg/kg daily, 800 mg/kg daily, 900 mg/kg daily, 1000 mg/kg daily, 1100 mg/kg daily, 1200 mg/kg daily, 1300 mg/kg daily, 1400 mg/kg daily, or 1500 mg/kg daily. In some embodiments, the FTI is administered at 1 mg/kg daily. In some embodiments, the FTI is administered at 2 mg/kg daily. In some embodiments, the FTI is administered at 5 mg/kg daily. In some embodiments, the FTI is administered at 10 mg/kg daily. In some embodiments, the FTI is administered at 20 mg/kg daily. In some embodiments, the FTI is administered at 50 mg/kg daily. In some embodiments, the FTI is administered at 100 mg/kg daily. In some embodiments, the FTI is administered at 200 mg/kg daily. In some embodiments, the FTI is administered at 500 mg/kg daily. The FTI can be administered either as a single dose or subdivided into more than one dose. In some embodiments, the FTI is tipifarnib.

In some embodiments, the FTI is administered at a dose of 50-2400 mg daily. In some embodiments, the FTI is administered at a dose of 100-1800 mg daily. In some embodiments, the FTI is administered at a dose of 100-1200 mg daily. In some embodiments, the FTI is administered at a dose of 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 1200 mg, or 2400 mg daily. In some embodiments, the FTI is administered at a dose of 200 mg daily. The FTI can be administered at a dose of 300 mg daily. The FTI can be administered at a dose of 400 mg daily. The FTI can be administered at a dose of 500 mg daily. The FTI can be administered at a dose of 600 mg daily. The FTI can be administered at a dose of 700 mg daily. The FTI can be administered at a dose of 800 mg daily. The FTI can be administered at a dose of 900 mg daily. The FTI can be administered at a dose of 1000 mg daily. The FTI can be administered at a dose of 1100 mg daily. The FTI can be administered at a dose of 1200 mg daily. The FTI can be administered at a dose of 1300 mg daily. The FTI can be administered at a dose of 1400 mg daily. The FTI can be administered at a dose of 1500 mg daily. The FTI can be administered at a dose of 1600 mg daily. The FTI can be administered at a dose of 1700 mg daily. The FTI can be administered at a dose of 1800 mg daily. The FTI can be administered at a dose of 1900 mg daily. The FTI can be administered at a dose of 2000 mg daily. The FTI can be administered at a dose of 2100 mg daily. The FTI can be administered at a dose of 2200 mg daily. The FTI can be administered at a dose of 2300 mg daily. The FTI can be administered at a dose of 2400 mg daily. The FTI can be administered either as a single dose or subdivided into more than one dose. In some embodiments, the FTI is tipifarnib.

In some embodiments, an FTI is administered at a dose of 100, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, or 1200 mg twice a day (b.i.d). In some embodiments, the FTI is administered at a dose of 100-1400 mg b.i.d. In some embodiments, the FTI is administered at a dose of 100-1200 mg b.i.d. In some embodiments, the FTI is administered at a dose of 300-1200 mg b.i.d. In some embodiments, the FTI is administered at a dose of 300-900 mg b.i.d. In some embodiments, the FTI is administered at a dose of 300 mg b.i.d. In some embodiments, the FTI is administered at a dose of 400 mg b.i.d. In some embodiments, the FTI is administered at a dose of 500 mg b.i.d. In some embodiments, the FTI is administered at a dose of 600 mg b.i.d. In some embodiments, the FTI is administered at a dose of 700 mg b.i.d. In some embodiments, the FTI is administered at a dose of 800 mg b.i.d. In some embodiments, the FTI is administered at a dose of 900 mg b.i.d. In some embodiments, the FTI is administered at a dose of 1000 mg b.i.d. In some embodiments, the FTI is administered at a dose of 1100 mg b.i.d. In some embodiments, the FTI is administered at a dose of 1200 mg b.i.d. In some embodiments, the FTI for use in the compositions and methods provided herein is tipifarnib.

As a person of ordinary skill in the art would understand, the dosage varies depending on the dosage form employed, condition and sensitivity of the patient, the route of administration, and other factors. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. During a treatment cycle, the daily dose could be varied. In some embodiments, a starting dosage can be titrated down within a treatment cycle. In some embodiments, a starting dosage can be titrated up within a treatment cycle. The final dosage can depend on the occurrence of dose limiting toxicity and other factors. In some embodiments, the FTI is administered at a starting dose of 300 mg daily and escalated to a maximum dose of 400 mg, 500 mg, 600 mg, 700 mg, 800 mg, 900 mg, 1000 mg, 1100 mg, or 1200 mg daily. In some embodiments, the FTI is administered at a starting dose of 400 mg daily and escalated to a maximum dose of 500 mg, 600 mg, 700 mg, 800 mg, 900 mg, 1000 mg, 1100 mg, or 1200 mg daily. In some embodiments, the FTI is administered at a starting dose of 500 mg daily and escalated to a maximum dose of 600 mg, 700 mg, 800 mg, 900 mg, 1000 mg, 1100 mg, or 1200 mg daily. In some embodiments, the FTI is administered at a starting dose of 600 mg daily and escalated to a maximum dose of 700 mg, 800 mg, 900 mg, 1000 mg, 1100 mg, or 1200 mg daily. In some embodiments, the FTI is administered at a starting dose of 700 mg daily and escalated to a maximum dose of 800 mg, 900 mg, 1000 mg, 1100 mg, or 1200 mg daily. In some embodiments, the FTI is administered at a starting dose of 800 mg daily and escalated to a maximum dose of 900 mg, 1000 mg, 1100 mg, or 1200 mg daily. In some embodiments, the FTI is administered at a starting dose of 900 mg daily and escalated to a maximum dose of 1000 mg, 1100 mg, or 1200 mg daily. The dose escalation can be done at once, or step wise. For example, a starting dose at 600 mg daily can be escalated to a final dose of 1000 mg daily by increasing by 100 mg per day over the course of 4 days, or by increasing by 200 mg per day over the course of 2 days, or by increasing by 400 mg at once. In some embodiments, the FTI is tipifarnib.

In some embodiments, the FTI is administered at a relatively high starting dose and titrated down to a lower dose depending on the patient response and other factors. In some embodiments, the FTI is administered at a starting dose of 1200 mg daily and reduced to a final dose of 1100 mg, 1000 mg, 900 mg, 800 mg, 700 mg, 600 mg, 500 mg, 400 mg, or 300 mg daily. In some embodiments, the FTI is administered at a starting dose of 1100 mg daily and reduced to a final dose of 1000 mg, 900 mg, 800 mg, 700 mg, 600 mg, 500 mg, 400 mg, or 300 mg daily. In some embodiments, the FTI is administered at a starting dose of 1000 mg daily and reduced to a final dose of 900 mg, 800 mg, 700 mg, 600 mg, 500 mg, 400 mg, or 300 mg daily. In some embodiments, the FTI is administered at a starting dose of 900 mg daily and reduced to a final dose of 800 mg, 700 mg, 600 mg, 500 mg, 400 mg, or 300 mg daily. In some embodiments, the FTI is administered at a starting dose of 800 mg daily and reduced to a final dose of 700 mg, 600 mg, 500 mg, 400 mg, or 300 mg daily. In some embodiments, the FTI is administered at a starting dose of 600 mg daily and reduced to a final dose of 500 mg, 400 mg, or 300 mg daily. The dose reduction can be done at once, or step wise. In some embodiments, the FTI is tipifarnib. For example, a starting dose at 900 mg daily can be reduced to a final dose of 600 mg daily by decreasing by 100 mg per day over the course of 3 days, or by decreasing by 300 mg at once. In some embodiments, the FTI is tipifarnib.

A treatment cycle can have different length. In some embodiments, a treatment cycle can be one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, a treatment cycle is 4 weeks. A treatment cycle can have intermittent schedule. In some embodiments, a 2-week treatment cycle can have 5-day dosing followed by 9-day rest. In some embodiments, a 2-week treatment cycle can have 6-day dosing followed by 8-day rest. In some embodiments, a 2-week treatment cycle can have 7-day dosing followed by 7-day rest. In some embodiments, a 2-week treatment cycle can have 8-day dosing followed by 6-day rest. In some embodiments, a 2-week treatment cycle can have 9-day dosing followed by 5-day rest. In some embodiment, a 4 week (28 day) treatment cycle can have 7 day dosing, followed by 21-day rest. In some embodiment, a 4 week (28 day) treatment cycle can have 21 day dosing, followed by 7-day rest. In some embodiment, a 4 week (28 day) treatment cycle can have dosing on days 1-7 and 15-21, and rest on days 8-14 and 22-28.

In some embodiments, FTI is administered on days 1-7 of a 28-day treatment cycle. In some embodiments, FTI is administered on days 1-7 and 15-21 of a 28-day treatment cycle. In some embodiments, FTI is administered on days 1-21 of a 28-day treatment cycle.

In some embodiments, the FTI can be administered for at least one treatment cycle. In some embodiments, the FTI can be administered for at least two, at least three, at least four, at least five, at least six, at least seven, at least eight, at least nine, at least ten, at least eleven, or at least twelve treatment cycles. In some embodiments, the FTI can be administered for at least two treatment cycles. In some embodiments, the FTI can be administered for at least three treatment cycles. In some embodiments, the FTI can be administered for at least six treatment cycles. In some embodiments, the FTI can be administered for at least nine treatment cycles. In some embodiments, the FTI can be administered for at least twelve treatment cycles. In some embodiments, the FTI is tipifarnib.

In some embodiments, the FTI is administered for up to two weeks. In some embodiments, the FTI is administered for up to three weeks, up to one month, up to two months, up to three months, up to four months, up to five months, up to six months, up to seven months, up to eight months, up to nine months, up to ten months, up to eleven months, or up to twelve months. In some embodiments, the FTI is administered for up to one month. In some embodiments, the FTI is administered for up to three months. In some embodiments, the FTI is administered for up to six months. In some embodiments, the FTI is administered for up to nine months. In some embodiments, the FTI is administered for up to twelve months.

In some embodiments, the FTI is administered daily for 3 of out of 4 weeks in repeated 4 week cycles. In some embodiments, the FTI is administered daily in alternate weeks (one week on, one week off) in repeated 4 week cycles. In some embodiments, the FTI is administered at a dose of 300 mg b.i.d. orally for 3 of out of 4 weeks in repeated 4 week cycles. In some embodiments, the FTI is administered at a dose of 600 mg b.i.d. orally for 3 of out of 4 weeks in repeated 4 week cycles. In some embodiments, the FTI is administered at a dose of 900 mg b.i.d. orally in alternate weeks (one week on, one week off) in repeated 4 week cycles. In some embodiments, the FTI is administered at a dose of 1200 mg b.i.d. orally in alternate weeks (days 1-7 and 15-21 of repeated 28-day cycles). In some embodiments, the FTI is administered at a dose of 1200 mg b.i.d. orally for days 1-5 and 15-19 out of repeated 28-day cycles.

In some embodiments, a 300 mg bid tipifarnib alternate week regimen can be used adopted. Under the regimen, patients receive a starting dose of 300 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some embodiments, a 600 mg bid tipifarnib alternate week regimen can be used adopted. Under the regimen, patients receive a starting dose of 600 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some embodiments, a 900 mg bid tipifarnib alternate week regimen can be used adopted. Under the regimen, patients receive a starting dose of 900 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some other embodiments, tipifarnib is given orally at a dose of 300 mg bid daily for 21 days, followed by 1 week of rest, in 28-day treatment cycles (21-day schedule; Cheng D T, et al., J Mol Diagn. (2015) 17(3):251-64). In some embodiments, a 5-day dosing ranging from 25 to 1300 mg bid followed by 9-day rest is adopted (5-day schedule; Zujewski J., J Clin Oncol., (2000) Feburary; 18(4):927-41). In some embodiments, a 7-day bid dosing followed by 7-day rest is adopted (7-day schedule; Lara P N Jr., Anticancer Drugs., (2005) 16(3):317-21; Kirschbaum M H, Leukemia., (2011) October; 25(10):1543-7). In the 7-day schedule, the patients can receive a starting dose of 300 mg bid with 300 mg dose escalations to a maximum planned dose of 1800 mg bid. In the 7-day schedule study, patients can also receive tipifarnib bid on days 1-7 and days 15-21 of 28-day cycles at doses up to 1600 mg bid.

In previous studies FTI were shown to inhibit the growth of mammalian tumors when administered as a twice daily dosing schedule. It was found that administration of an FTI in a single dose daily for one to five days produced a marked suppression of tumor growth lasting out to at least 21 days. In some embodiments, FTI is administered at a dosage range of 50-400 mg/kg. In some embodiments, FTI is administered at 200 mg/kg. Dosing regimen for specific FTIs are also well known in the art (e.g., U.S. Pat. No. 6,838,467, which is incorporated herein by reference in its entirety). For example, suitable dosages for the compounds Arglabin (WO98/28303), perrilyl alcohol (WO 99/45712), SCH-66336 (U.S. Pat. No. 5,874,442), L778123 (WO 00/01691),2(S)-[2(S)-[2(R)-amino-3-mercapto]propylamino-3(S)-methyl]-pentyloxy-3-phenylpropionyl-methionine sulfone (WO94/10138), BMS 214662 (WO 97/30992), AZD3409; Pfizer compounds A and B (WO 00/12499 and WO 00/12498) are given in the aforementioned patent specifications which are incorporated herein by reference or are known to or can be readily determined by a person skilled in the art.

In relation to perrilyl alcohol, the medicament may be administered 1-4 g per day per 150 lb human patient. In one embodiment, 1-2 g per day per 150 lb human patient. SCH-66336 typically may be administered in a unit dose of about 0.1 mg to 100 mg, more preferably from about 1 mg to 300 mg according to the particular application. Compounds L778123 and 1-(3-chlorophenyl)-4-[1-(4-cyanobenzyl)-5-imidazolylmethyl]-2-piperazinone may be administered to a human patient in an amount between about 0.1 mg/kg of body weight to about 20 mg/kg of body weight per day, preferably between 0.5 mg/kg of bodyweight to about 10 mg/kg of body weight per day.

Pfizer compounds A and B may be administered in dosages ranging from about 1.0 mg up to about 500 mg per day, preferably from about 1 to about 100 mg per day in single or divided (i.e. multiple) doses. Therapeutic compounds will ordinarily be administered in daily dosages ranging from about 0.01 to about 10 mg per kg body weight per day, in single or divided doses. BMS 214662 may be administered in a dosage range of about 0.05 to 200 mg/kg/day, preferably less than 100 mg/kg/day in a single dose or in 2 to 4 divided doses.

A person of ordinary skill in the art would understand that the methods described herein include using any permutation or combination of the specific FTI, formulation, dosing regimen, additional therapy to treat a subject described herein.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

Throughout this application various publications have been referenced. The disclosures of these publications in their entireties, including GenBank and GI number publications, are hereby incorporated by reference in this application in order to more fully describe the state of the art to which this disclosure pertains. Although the invention has been described with reference to the examples provided above, it should be understood that various modifications can be made without departing from the spirit of the invention.

### EXAMPLES

A Phase II study was performed that investigated the antitumor activity, in terms of objective response rate, of tipifarnib in subjects with locally advanced, unresectable or metastatic, relapsed and/or refractory tumors that carry HRAS mutations and for whom there is no curative therapy available. Subjects were men and women over 18 years of age.

Inclusion criteria of the study were as follows: histologically or cytologically confirmed diagnosis of thyroid cancer (cohort 1) or squamous head and neck cancer (cohort 2) for which there is no curative therapy available; tumor that carries a missense HRAS mutation; subject consents to provide at least 10 unstained tumor slides for retrospective testing of HRAS gene tumor status; subject has measurable disease according to RECIST v1.1 and has relapsed or is refractory to prior therapy; at least 2 weeks since the last systemic therapy or radiotherapy regimen prior to enrolment; ECOG PS 0 or 1; acceptable liver function; acceptable renal function; and acceptable hematologic status.

Exclusion criteria of the study were as follows: prior treatment with an FTase inhibitor; history of relevant coronary heart disease or myocardial infarction within last 3 years, NYHA Grade III or greater congestive heart failure, cerebro-vascular attack within the prior year, or serious cardiac arrhythmia requiring medication except atrial fibrillation; known uncontrolled brain, leptomeningeal or epidural metastases (unless treated and well controlled for at least 4 weeks prior to Cycle 1 Day 1); controlled brain metastases that require continuous high dose corticosteroid use within 4 weeks of Day 1; non-tolerable>Grade 2 neuropathy or evidence of unstable neurological symptoms within 4 weeks first dose; major surgery, other than diagnostic surgery, within 4 weeks prior to first dose, without complete recovery; active, uncontrolled bacterial, viral, or fungal infections, requiring systemic therapy; or known infection with HIV, or an active infection with hepatitis B or hepatitis C.

The study comprised 36 participants and subjects were enrolled into two nonrandomized cohorts as follows: Cohort 1 was comprised of patients with malignant thyroid tumors with HRAS mutations; Cohort 2 was comprised of patients with squamous head and neck cancer with HRAS mutations. Patients received tipifarnib at a starting dose of 600-900 mg orally twice daily on days 1-7 and 15-21 of 28-day cycles.

The primary outcome measurement of the study was objective response rate. Additionally, progression-free survival, duration of response, and the number of subjects with adverse events and serious adverse events were also recorded as secondary outcome measures. Adverse events and serious adverse events were graded according to the NCI-CTCAE (Version 4.03).

Upon meeting the primary objective (objective response rate), the study was amended to continue enrollment of HNSCC patients (Cohort 2) in addition to patients with other SCCs (Cohort 3). These patients had tumors carrying high HRASm variant allele frequency (VAF >35% or if baseline serum albumin is >3.5 g/dL)

Twenty-three patients with HNSCC and 10 patients with different SCC were been treated. Patients had relapsed/refractory disease with a median of 2 prior regimens and no objective partial responses observed on their last prior therapy including platinum, immunotherapy and cetuximab+/−chemotherapy. Of the 15 HNSCC patients meeting HRASm VAF criteria, a 53% overall response rate was observed with 8 confirmed partial responses (PR) and 5 stable disease (SD). Two patients had not yet received the first on-study tumor response assessment.

The overall median progression-free survival (PFS) was 5.4 months (95% CI 4.5 to 19.5 months, N=15); 19 months (95% CI 5.3 to 19.5 months) for patients experiencing a PR and 4.5 months (95% CI 1.6 to 5.4 months) for those experiencing SD. The median time of PFS on last prior therapy was 3.2 months. All patients had at least 1 treatment-emergent adverse event (TEAE). The most frequently observed drug-related Grade >3 TEAEs occurring in ≥10% patients were blood and lymphatic system disorders, gastrointestinal disorders, and renal disorders.

We claim:

1. A method of treating a subject having an H-Ras mutant squamous cell carcinoma (SCC) in conjunction with surgery, comprising administering a therapeutically effective amount of a farnesyltransferase inhibitor (FTI) to the subject, wherein the SCC has an H-Ras mutant allele frequency (AF) that is greater than 20%.

2. The method of claim 1, wherein the H-Ras mutant AF is greater than 25%, 30%, 35%, 40%, 45%, 50%, 55%, or 60%.

3. The method of claim 1 or 2, wherein the SCC has an amino acid substitution in H-Ras at a codon selected from a group consisting of G12, G13, Q61, Q22, K117, A146, and any combination thereof.

4. The method of any one of claims 1 to 3, wherein the SCC does not have K-Ras mutation or N-Ras mutation.

5. The method of any one of claims 1 to 4, further comprising administering radiation therapy or chemotherapy to the subject.

6. The method of any one of claims 1 to 5, wherein the FTI is administered before the surgery.

7. The method of claim 6, wherein the FTI reduces tumor burden in the subject by at least 20%, at least 30%, at least 40%, at least 50%, at least 60%, at least 70%, at least 80%, at least 90%, or at least 95%.

8. The method of claim 6, wherein the FTI converts the SCC from inoperable to operable.

9. The method of any one of claims 1 to 5, wherein the FTI is administered after the surgery.

10. The method of claim 9, wherein the FTI reduces the risk of recurrence after the surgery.

11. The method of any one of claims 1 to 10, wherein the SCC is head and neck SCC (HNSCC), lung SCC (LSCC), thyroid SCC (TSCC), esophageal SCC (ESCC), bladder SCC (BSCC), cervical SCC, vulvar SCC, penile SCC, cutaneous SCC or urothelial carcinoma (UC).

12. The method of claim 11, wherein the SCC is HNSCC.

13. The method of claim 12, wherein the HNSCC is HNSCC of the trachea.

14. The method of claim 12, wherein the HNSCC is HNSCC of the maxilla.

15. The method of claim 12, wherein the HNSCC is HNSCC of the oral cavity.

16. The method of any one of claims 1 to 15, wherein the SCC is human papillomavirus (HPV)-negative.

17. The method of any one of claims 1 to 15, wherein the SCC is at an advanced stage or metastatic.

18. The method of any one of claims 1 to 15, wherein the SCC is relapsed.

19. The method of any one of claims 1 to 15, wherein the SCC is refractory.

20. The method of any one of claims 1 to 19, comprising determining the H-Ras mutant AF in a sample from the subject.

21. The method of claim 20, wherein said H-Ras mutant AF is determined by sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay.

22. The method of claim 21, wherein the sample is a tissue biopsy.

23. The method of claim 21, wherein the sample is a tumor biopsy.

24. The method of claim 21, wherein the sample is a blood sample.

25. The method of claim 21, wherein the sample is isolated cells.

26. The method of any one of claims 1 to 25, wherein the FTI is selected from the group consisting of tipifarnib, lonafarnib, arglabin, perrilyl alcohol, L778123, L739749, FTI-277, L744832, CP-609,754, 8208176, AZD3409, and BMS-214662.

27. The method of claim 26, wherein the FTI is tipifarnib.

28. The method of claim 26, wherein the FTI is lonafarnib.

29. The method of claim 26, wherein the FTI is BMS-214662.

30. The method of any one of claims 1 to 29, wherein the FTI is administered at a dose of 0.05-500 mg/kg body weight.

31. The method of any one of claims 1 to 29, wherein the FTI is administered at a daily dose of 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg.

32. The method of any one of claims 1 to 29, wherein the FTI is administered twice a day.

33. The method of claim 32, wherein the FTI is administered at a dose of 100-1000 mg twice a day.

34. The method of claim 32, wherein the FTI is administered at a dose of 100 mg, 200 mg, 300 mg, 400 mg, 500 mg, 600 mg, 800 mg, 900 mg, or 1000 mg twice a day.

35. The method of any one of claims 1 to 34, comprising administering the FTI for a period of one to seven days.

36. The method of claim 35, wherein the FTI is administered on days 1-7 of a 28-day treatment cycle.

37. The method of claim 35, wherein the FTI is administered on days 1-7 and 15-21 of a 28-day treatment cycle.

38. The method of claim 35, wherein the FTI is administered on days 1-21 of a 28-day treatment cycle.

39. The method of any one of claims 35 to 38, wherein the FTI is administered for at least 3 cycles, at least 6 cycles, at least 9 cycles, or at least 12 cycles.
